(19) United States
(12) Patent Application Publication
(10) Pub. No.: US 2015/0272591 A1
(43) Pub. Date: Oct. 1, 2015

(54) DEVICES AND METHODS FOR MODIFYING A VOLUME OF A CAVITY

(71) Applicant: Boston Scientific Scimed, Inc., Maple Grove, MN (US)

(72) Inventors: Martyn G. FOLAN, Galway (IE); Fergal HORGAN, Co. Mayo (IE); Michael G. HAYES, Galway (IE); Javier PALOMAR-MORENO, Galway (IE); Patricia KELLY, Galway (IE); Man Minh NGUYEN, Harvard, MA (US); Kevin John WILCOX, Brighton, MA (US)

(73) Assignee: Boston Scientific Scimed, Inc.

(21) Appl. No.: 14/672,597

(22) Filed: Mar. 30, 2015

Related U.S. Application Data
(60) Provisional application No. 61/972,671, filed on Mar. 31, 2014.

(51) Int. Cl. A61B 17/12

(52) U.S. Cl. CPC A61B 17/12181; A61B 17/12104; A61B 17/12036; A61B 2017/00022

(57) ABSTRACT

Exemplary embodiments of a method for assisting breathing in a lung having damaged tissue are disclosed. The method may include inserting an implant containing a dilatant fluid into a pleural cavity of a patient to reduce an inhalation volume of the lung. Additional embodiments may include an implant configured to apply a force toward a portion of a lung. A controller may be coupled to the implant and configured to sense a biological event and control an amount of outwardly directed force applied by the implant to the lung in response to the biological event. Further, a method may include applying a force to an organ via an implant to expel material from the organ and adjusting the implant so as to transition between a first configuration and a second configuration. The implant may include at least one of an electro-active material and a pressure sensitive material.

### CROSS-REFERENCE TO RELATED APPLICATION

This patent application claims the benefit of priority to U.S. Provisional Patent Application No. 61/972,671, filed on Mar. 31, 2014, the entirety of which is incorporated herein by reference.

### TECHNICAL FIELD

Embodiments of the present disclosure relate to methods and systems for improving organ function within a patient's body. In one embodiment, for example, a device may include a space occupying device configured to exert a pressure upon an organ. In further embodiments, the disclosed devices and methods may relate to treating the pulmonary system for various problems, defects, diseases, including, but not limited to, chronic obstructive pulmonary disease (COPD). Additional pulmonary disorders may include emphysema, asthma, and/or bronchitis. More particularly, the present disclosure relates to devices, systems, and methods for assisting breathing in a lung having damaged tissue by either preventing air from contacting certain portions of the lung and/or expelling air from certain portions of the lung by, for example, exerting a force against portions of the lung to compress these portions.

### BACKGROUND

Chronic obstructive pulmonary disease (COPD) is a serious progressive lung disease, which makes it harder to breathe. COPD may include chronic bronchitis and/or emphysema, a pair of commonly co-existing diseases of the lungs in which the airways may narrow over time. This may limit airflow to and from the lungs, causing, among other things, shortness of breath (dyspnea). COPD is typically irreversible and worsens over time. Some management strategies for treating COPD include, but are not limited to, smoking cessation, vaccinations, rehabilitation, and drug therapy (e.g., inhalers or oral pharmaceuticals). Emphysema, a type of COPD, is a long-term lung disease. In a patient suffering from emphysema, the tissues necessary to support the physical shape and function of the lungs are destroyed, the tissue loses its elasticity, or is otherwise damaged. The patient may not be able to expel the trapped air in the diseased lung, and the lung may quickly become filled with stagnant air.

It may, therefore, be beneficial to provide a technique of treating or appropriately manipulating airways of the lungs for treating COPD as well as other pulmonary issues, problems, defects, diseases, etc.

### SUMMARY

Embodiments of the present disclosure relate to methods and systems for improving organ function within a patient's body. In one embodiment, for example, a device may include a space occupying device configured to exert a pressure upon an organ. The disclosed embodiments relate particularly to methods and devices for assisting breathing in a lung, for example, a lung having damaged tissue, and for expelling air from the lung.

One exemplary embodiment may include a method for assisting breathing in a lung having damaged tissue. The method may include inserting an implant containing a dilatant fluid into a pleural cavity of a patient to reduce an inhalation volume of the lung.

The exemplary method may further include one or more of the following features: the damaged tissue may be an emphysematous portion of the lung; the method may further include inserting the implant adjacent to the damaged tissue to prevent the damaged tissue from expanding during inhalation; the implant and dilatant fluid may exert a force on the damaged tissue during exhalation of the lung; the implant may be resilient; and wherein the dilatant fluid may include one or more of hyaluronic acid, lubricin, cornstarch, water, Dilatal, acrylic acid, ester-styrene, silicone oil, boric acid, and ethylene glycol.

An additional exemplary embodiment may include a system. The medical device may include an implant for insertion into a lung cavity. The implant may be configured to apply an outwardly directed force toward a portion of a lung disposed in the lung cavity. The medical device may further include a controller coupled to the implant. The controller may be configured to sense a biological event and control an amount of outwardly directed force applied by the implant to the lung in response to the biological event.

The exemplary medical device may further include one or more of the following features: the biological event may be dyspnea or an irregular heartbeat; the controller may be further configured to increase the amount of outwardly directed force applied by the implant to the lung in response to the biological event; the controller may be further configured to decrease the amount of outwardly directed force applied by the implant to the lung after the cessation of the biological event; the biological event may be one of an inhalation and exhalation rhythm of the lung; the controller may be further configured to synchronize the outwardly directed force applied by the implant to the lung by the one of the inhalation and exhalation rhythm; the controller may be configured to increase the outwardly directed force applied by the implant to the lung during exhalation of the lung; the controller may be configured to decrease the outwardly directed force applied by the implant to the lung during inhalation of the lung; and the implant may include an anti-migration element.

A further exemplary embodiment may include a method of assisting in evacuating an interior volume of an organ. The method may include applying a force to the organ via an adjustable implant placed adjacent to the organ to assist in evacuating material from the interior volume of the organ. The method may further include adjusting the implant so as to transition between a first configuration and a second configuration. Additionally, the implant may include at least one of an electro-active material and a pressure sensitive material.

This exemplary method may further include one or more of the following features: if the implant includes an electro-active material, the method may further include applying an electrical charge to cause the implant to transition between the first and second configurations; the implant may be collapsed in the first configuration and may be expanded in the second configuration; the cavity may be a lung and the material may be air, and the electrical charge may be applied during exhalation of the lung; if the implant includes a pressure sensitive material, the cavity may be a lung and the material may be air, and the pressure sensitive material may be configured to collapse during inhalation of the lung and expand during exhalation of the lung; and the implant may further include a spring, a stent, or a polymer.

The above summary of exemplary embodiments is not intended to describe each disclosed embodiment or every implementation of the present disclosure. The Figures, and Detailed Description, which follow, more particularly exemplify these exemplary embodiments.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 illustrates an exemplary expandable system deployed within a patient;

FIG. 2 illustrates the exemplary expandable system of FIG. 1 adjacent a lung in an expanded configuration;

FIGS. 3 & 4 illustrate another exemplary expandable implant deployed in a pleural cavity of a patient;

FIG. 5 illustrates yet another alternative exemplary implant deployed in a patient;

FIG. 6 illustrates a further exemplary implant deployed within a patient; and

FIG. 7 illustrates yet another exemplary implant deployed within a patient.

### DETAILED DESCRIPTION OF EXEMPLARY EMBODIMENTS

Reference will now be made in detail to exemplary embodiments of the present disclosure, examples of which are illustrated in the accompanying drawings. Wherever possible, the same reference numbers will be used throughout the drawings to refer to the same or like parts or components. The term “distal” refers to the direction that is away from the user/operator or medical professional and into the patient's body. By contrast, the term “proximal” refers to the direction that is closer to the user/operator/medical professional and away from the patient's body.

Exemplary embodiments of the present disclosure relate to medical devices/implants/expandable systems and methods for assisting breathing in a lung having damaged or diseased tissue. The implants may be, for example, inserted into a pleural cavity of a patient to reduce an inhalation volume of the lung. The implant may contain, for example, dilatant fluid, saline, gas, silicone, and so forth. The damaged tissue may be an emphysematous portion of the lung. Insertion of the implant adjacent to the damaged tissue may prevent the damaged tissue from expanding during inhalation. The implant may also exert a force on the damaged tissue during exhalation of the lung. The implant may be a sponge, balloon, pouch, spring, boost device, and so forth that may be inserted to reduce an inhalation volume of the lung.

### Exemplary Embodiments

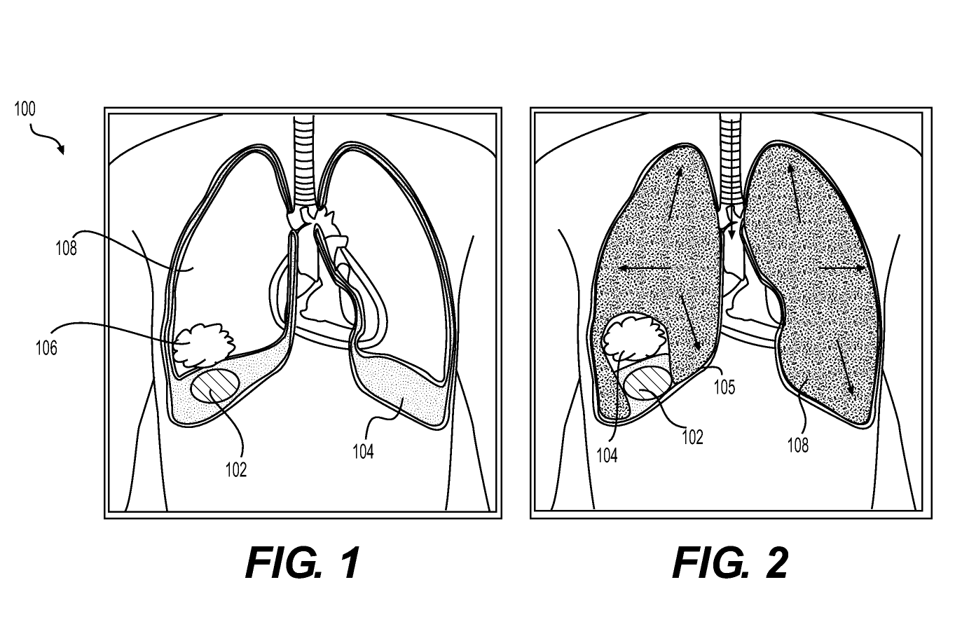

FIG. 1 illustrates a lung system 100 with an exemplary expandable system 102 (also referred to herein as an implant). As shown in FIG. 1, for example, the expandable system 102 may be deployed in a pleural cavity 104, external of a lung, for treating an unhealthy target region or damaged tissue 106 of the lung according to a first embodiment of the present disclosure. While described herein with reference to lung system 100, it is understood that expandable system 102 may be deployed in any system so as to fill a cavity adjacent any organ within the patient's body.

When a person inhales, air flows in through the nose and/or mouth of the person, and a trachea coupled to the lung system 100 delivers the air to lungs of the lung system 100 for respiratory functions. The trachea leads to a number of bronchial passages or airways and terminates in a plurality of alveoli. The alveoli are small elastic air sacs which enable gas exchange. That is, they permit oxygen diffusion into the blood stream, and receive and expel CO₂ during exhalation.

During inhalation, air is delivered to the lungs (or the lung system 100) and is received within the alveoli via bronchial passages or airways. The air inflates the alveoli, which later recoil to exhale air. This operation of the lungs during the inhalation and exhalation of air may be disturbed due to certain malfunctions, injuries, defects, or diseases, such as chronic obstructive pulmonary disease (COPD), including, but not limited to, chronic bronchitis and/or emphysema.

As shown in FIG. 1, the lung system 100 of a patient may include lungs having healthy tissue 108 and damaged tissue 106. The damaged tissue 106 may be an emphysematous portion of the lung system 100. The expandable system 102 may be inserted into the pleural cavity 104 adjacent to the damaged tissue 106 to prevent the damaged tissue 106 from expanding during inhalation, thereby reducing an inhalation volume of the lung system 100.

In one embodiment, the expandable system 102 may be configured to apply an outwardly directed force (relative to the expandable system 102) to the lungs. The expandable system 102 may include, for example, an inflatable member (e.g., balloon) configured to be strategically placed external to the lungs, in the pleural cavity 104. The expandable system 102 may be comprised of any suitable material(s) such as, but not limited to, PTFE, Nylon, PEBAX®, latex, or similar, and/or combinations of these materials. Additionally or alternatively, the expandable system 102 may include suitable radiopaque marking or may be impregnated with radiopaque materials to facilitate visualization within the body. The disclosed expandable system 102 may be formed through various suitable methods such as, for example, extrusion and so forth. In some embodiments, the expandable system 102 (and/or any disclosed expandable system herein) may include a lubricous coating to reduce potential friction due to expansion and contraction of the lungs and associated muscle groups, for example, intercostal and diaphragm tissue and muscles.

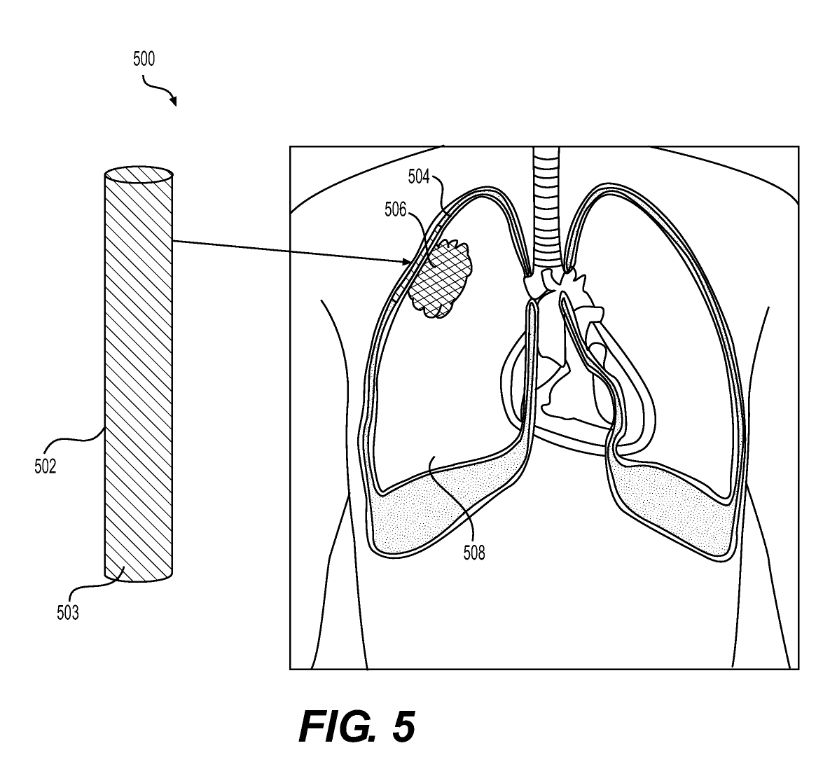

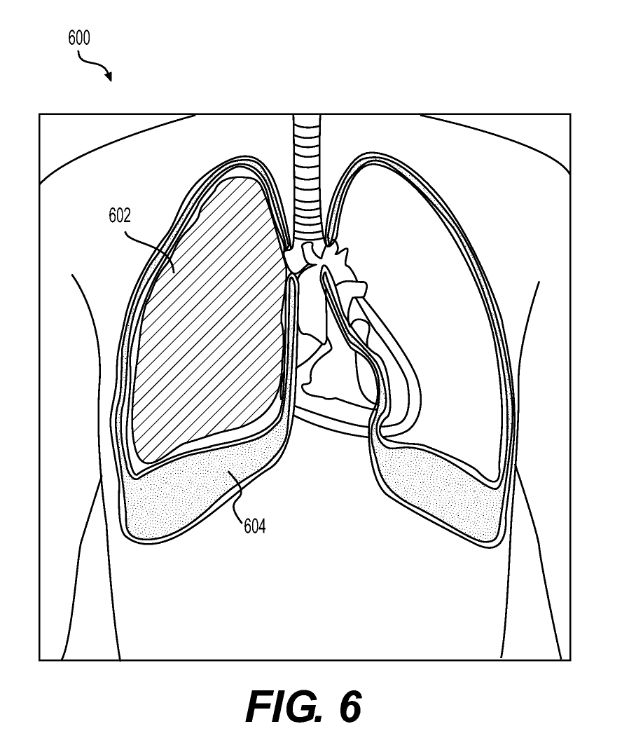

Further, the internal volume of the expandable system 102 may be filled with a suitable material including, for example, water, saline, gas, air, and/or a gel such as, for example, silicone. Additionally or alternatively, expandable system 102 may be filled with dilatant fluids as explained in further detail with respect to FIG. 5. A dilatant fluid (also referred to as shear-thickening fluid) is typically a non-Newtonian fluid in which viscosity increases with the rate of shear strain or pressure.

In one exemplary embodiment, the expandable system 102 may be a polymeric balloon filled with a suitable material such as dilatant fluid. When used in the expandable system 102, the viscous gradient demonstrated by the dilatant fluid may act as a hard stop for damaged tissue 106. In this way, the dilatant fluid may inhibit the damaged tissue 106 from expanding beyond a predetermined amount and taking in inhaled air while permitting the healthy tissue 108 to expand as normal.

In an embodiment, the expandable system 102 may be implanted into the pleural cavity 104 between the base of the lung(s) and the diaphragm as shown in FIG. 1. In another embodiment, the expandable system 102 can be implanted into the pleural cavity 104 between a defined area of a lung lobe and the ribcage (as shown in FIG. 5).

Further, the expandable system 102 may be delivered into the pleural cavity 104 using a suitable delivery system such as a laparoscopic or endoscopic delivery system (not shown) and/or any other minimally invasive system or procedure. In at least one embodiment, the delivery system may be similar to a standard vascular or luminal access system or the like. In a collapsed state, the expandable system 102 may be mounted on the delivery device for delivery of the expandable system 102 adjacent to the damaged or diseased tissue 106 as identified via preliminary CT scan information detailing the size and nature of damaged tissue 106 or the emphysematous region.

In some embodiments, the expandable system 102 may be delivered into the pleural cavity 104 in a collapsed state. Once positioned at a target region (e.g., adjacent to the damaged tissue 106), the expandable system 102 may be deployed (e.g., inflated, filled, and/or expanded) using any suitable mechanism. During deployment, the expandable system 102 may be inflated by filling the expandable system 102 with a suitable volume of material and then sealed or otherwise maintained in the expanded state. The material may include, but is not limited to, a fluid, such as a liquid or a gas, or a combination of these. The liquid or gas may be injected or otherwise delivered into the expandable system 102 in the collapsed state after delivery of the expandable system 102 to the pleural cavity 104. In some embodiments, the expandable system 102 and/or the liquid and/or gas can be delivered using an augmented vascular or luminal access type device that implants the expandable system 102 in the desired location. The access site can then be sealed in a manner similar to that used for standard vascular or luminal access procedures. In some embodiments, the expandable system 102 may be delivered into the pleural cavity 104 in an expanded state via, for example, open surgery.

In some embodiments, the size of the expandable system 102 may be adjusted during or after implantation by varying the volume of material inserted into the expandable system 102 by the operating clinician or medical professional. In at least one embodiment, the patient's breathing may be monitored (e.g., via spirometry) to ascertain the correct fit, location, and size of the expandable system 102 to maximize the impact of the implanted expandable system 102. For example, spirometric measures of pulmonary function may be used to gauge size and location for efficacy to treatment. Accordingly, the expansion and contraction pattern of the lungs may be monitored to ascertain the proper fit and positioning for expandable system 102.

In alternate embodiments, the expandable system 102 can be sized to determine correct fit post implantation of the expandable system 102. Therefore, in this embodiment, if the damaged tissue 106 increases in size, an operator may be able to inject additional material to increase the size of the expandable system 102. The operator may accomplish this through an access port or the like. In some embodiments, the port may be disposed under the skin at a location remote from the expandable system 102 but operably coupled to the interior of the expandable system 102.

FIG. 2 illustrates the exemplary lung system 100 with implanted expandable system 102 of FIG. 1 after inhalation of air by a patient. As shown, the expandable system 102 compresses and thereby prevents the damaged tissue 106 from expanding after inhalation of air in the lungs. At the same time, the healthy tissue 108 expands normally as patient inhales air. That is, the expandable system 102 may inhibit the damaged tissue 106 of the lung system 100 from expanding while permitting the healthy tissue 108 of the lung system 100 to expand to its full potential, enabling effective respiration and reducing the potential for air trapping in the damaged tissue 106 of the lung system 100. The patient may then experience more typical breathing patterns, thereby improving mobility without the need for additional invasive supplemental oxygen or other treatments. Additionally, the disclosed expandable system 102 reduces the potential for COPD related exacerbations which have further detrimental effects on the patient well-being and disease progression. As explained in further detail below, in some embodiments, a controller (not shown) may be disposed on the expandable system 102. In further embodiments, the controller may be external to the patient's body and configured to control or regulate (e.g., remotely) the expandable system 102.

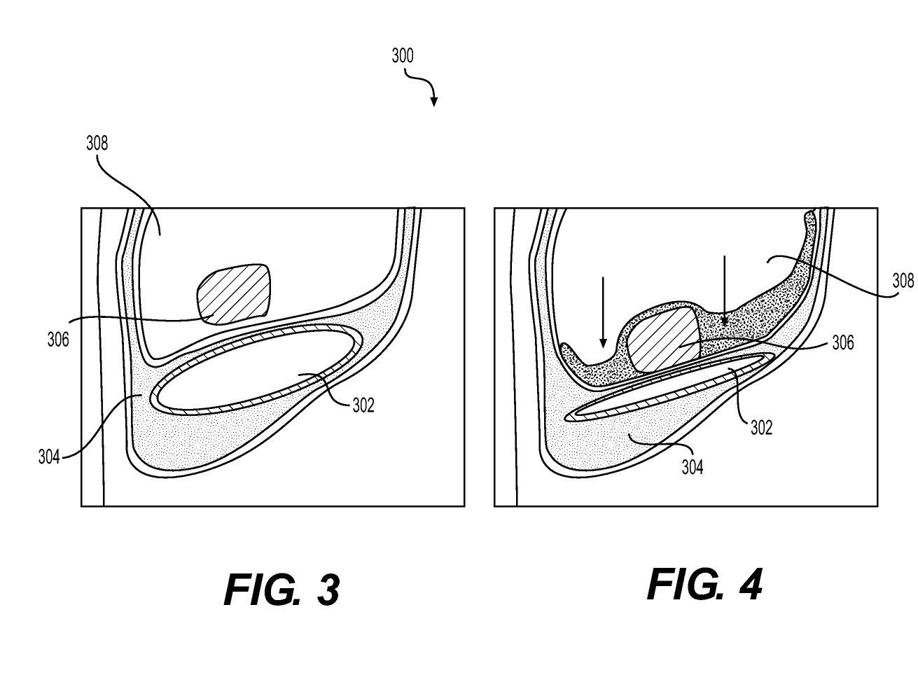

FIGS. 3 & 4 illustrate an exemplary expandable system 302 deployed in a pleural cavity 304 of a lung system 300. The lung system 300 of a patient suffering from COPD may include an emphysematous or otherwise damaged region including damaged tissue 306, and a healthy region including healthy tissue 308. As shown, the expandable system 302 may be a mechanically expandable assembly such as, e.g., a sponge which may be inserted into the pleural cavity 304 of the lung system 300 to reduce an inhalation volume of the lung and in turn, to treat the lung for injury, defect, or disease, such as emphysema.

For treating the damaged tissue 306, a medical device including the expandable system 302 can be inserted into a lung cavity of the lung system 300. The medical device may be deployed in the pleural cavity 304 and external to the lungs. The expandable system 302 may be configured to apply an outwardly directed force (relative to expandable system 302) toward a lung disposed in the lung cavity or the lung system 300 when, e.g., a lung expands during inhalation. In some embodiments, a controller (not shown) may positioned internally or externally to the patient's body and may be configured to control (e.g., remotely) the contraction and expansion of the expandable system 302.

The expandable system 302 may be formed as a sponge, using various suitable polymers. The polymer selected may be memory foam, such as, for example, polyurethane and additional chemicals to increase its viscosity and density. This material is often referred to as a “visco-elastic” polyurethane foam or low-resilience polyurethane foam (LRPu). Polyurethane foam is low-density memory foam that is pressure-sensitive and molds quickly to the shape of a body part pressing against it, returning to its original shape once the pressure is removed. The sponge optionally may be enclosed within an outer member comprised of, for example, PEBAX or silicone.

The expandable system 302 may be formed through extrusion or other suitable methods. Further, the expandable system 302 may be delivered minimally invasively into the pleural cavity 304 using an augmented vascular or luminal access type device that may implant the expandable system 302 in the desired location. In at least one embodiment, the expandable system 302 including a sponge may be premounted on the delivery device and then may be deployed at the desired location adjacent or in accordance to the damaged tissue 306 of the pleural cavity 304. Then, the expandable system 302 may be filled with a suitable material as required to the correct volume and sealed or otherwise maintained in an expanded state. The access site can then be sealed in a manner similar to that used for vascular or luminal access. That is, in some embodiments, the sponge may include an internal bladder that may be selectively filled to adjust sizing of and/or stiffness of the sponge.

Further, the size, shape, and/or density of the expandable system 302 may vary depending on the size of the damaged tissue 306. In at least one embodiment, the expandable system 302 can be sized during implantation by the operating clinician. As noted above, the patient's breathing mechanisms also may be cycled to ascertain the correct location and size of the sponge based on observation to maximize the impact of the expandable system 302.

Turning now to FIG. 4, the sponge of the expandable system 302 may be resilient in construction and may compress due to pressure of air in the lung system 300. After successful implantation of the sponge upon inhalation by the patient, the lungs (or the lung system 300) may expand normally in response to the reduction in pressure caused by the cavity formed by outward movement of the chest muscles, i.e., diaphragm and intercostal muscles and tissue. This in turn may cause the implanted expandable system 302 to be crushed/pushed down into a compact volume. With subsequent exhalation, the healthy tissue 308 of the lung system 300 collapses back in on itself, expelling air from the lungs. The damaged tissue 306, as already discussed, has a tendency not to collapse during exhalation due to a loss of elasticity. After collapse of the healthy tissue 308 however, the expandable system 302 is exposed to reduced compression forces and therefore, the expandable system 302 can return to its initial shape. That is, due to the expandable system's 302 resilient nature (e.g., sponge), upon removal of compression forces, the expandable system 302 may return to its expanded initial shape. During exhalation, the implanted expandable system 302 may cause the inelastic regions of the damaged tissue 306 to collapse in along with the natural collapse of the healthy tissue 308 and force additional trapped air out of the lung system 300, thus reducing the residual air within the lung system 300. As such, the expandable system 302 may assist in the reduction of air trapped in the damaged tissue 306, which may otherwise build up with each subsequent breath leaving the patient with effectively no breathing potential after a short time interval. The patient can then return to a more typical mobility without the need for additional invasive supplemental oxygen or other treatments. The expandable system 302 including a sponge may reduce the potential for COPD related exacerbations which may have further detrimental effects on the patient's well-being and disease progression.

FIG. 5 illustrates another alternative exemplary expandable system 502 deployed in a pleural cavity 504 of a lung system 500 according to a third embodiment of the present disclosure. In at least one embodiment, the expandable system 502 may be a balloon system similar to the expandable system 102 as disclosed in FIGS. 1 and 2. In alternate embodiments, the expandable system 502 may be a mechanical assembly, e.g., a sponge system similar to the expandable system 302 disclosed in FIGS. 3 and 4. In either embodiment, the expandable system 502 may be implanted into the pleural cavity 504 between a defined area of a lung lobe and a patient's ribcage. Additionally, the expandable system 502 may be delivered within the patient's body using similar methods/systems as described above.

Further, the shape, size, and location of the expandable system 502 may vary depending on the size and shape of damaged tissue 506. For example, in some embodiments, the expandable system 502 (or any disclosed expandable system) may be substantially spherical, cylindrical, rectangular, planar, etc. The expandable system 502 may be formed using suitable material/polymer, such as, but not limited to, PTFE, Nylon, PEBAX®, latex, or similar mixtures of the same. Further, the disclosed expandable system 502 may be formed using suitable methods, such as, extrusion. In some embodiments, the internal volume of the expandable system 502 may be filled with a suitable material. For example, the internal volume of the expandable system 502 may be filled with a suitable material such as a dilatant fluid 503. The dilatant fluid 503 (also referred to as shear-thickening fluid) is a non-Newtonian fluid in which viscosity increases with the rate of shear strain. Typically, such a fluid may include a colloid suspension. The increased viscosity behavior of the fluid is observed because the fluid crystallizes under stress and behaves more like a solid than a fluid. Thus, the viscosity of a shear-thickening fluid is dependent on the shear rate it experiences. Additionally or alternatively, a Newtonian fluid configured to exhibit non-Newtonian behavior, e.g., cornstarch in water (Oobleck), may be used. Exemplary dilatant fluids 503 may include or be similar in design to synovial fluid (e.g., hyaluronic acid and lubricin), Oobleck (cornstarch/water), Dilatal™ (BASF), acrylic acid ester-styrene/water, Silly Putty (silicone oil/boric acid), and silica/poly (ethylene glycol). FIG. 6 illustrates another alternative, exemplary expandable system 602 deployed in a pleural cavity 604 of lung system 600, according to a fourth embodiment of the present disclosure. The lung system 600 includes at least two lungs having multiple lung lobes. As shown, the expandable system 602 can be deployed into the pleural cavity 604 adjacent to an entire area of one or both the lungs which incorporates multiple lung lobes. The expandable system 602 may be a sponge system or a balloon system similar to the expandable system 302 and the expandable system 102 as described above. In such an embodiment, the lung may suffer from heterogeneous disease within one or more pockets of the lung dispersed across the total lung volume. By placement of the expandable system 602 across or behind an entire lung, the expandable system 602 may prevent weaker portions of the lung from expanding while allowing healthier stronger portions to expand during inhalation.

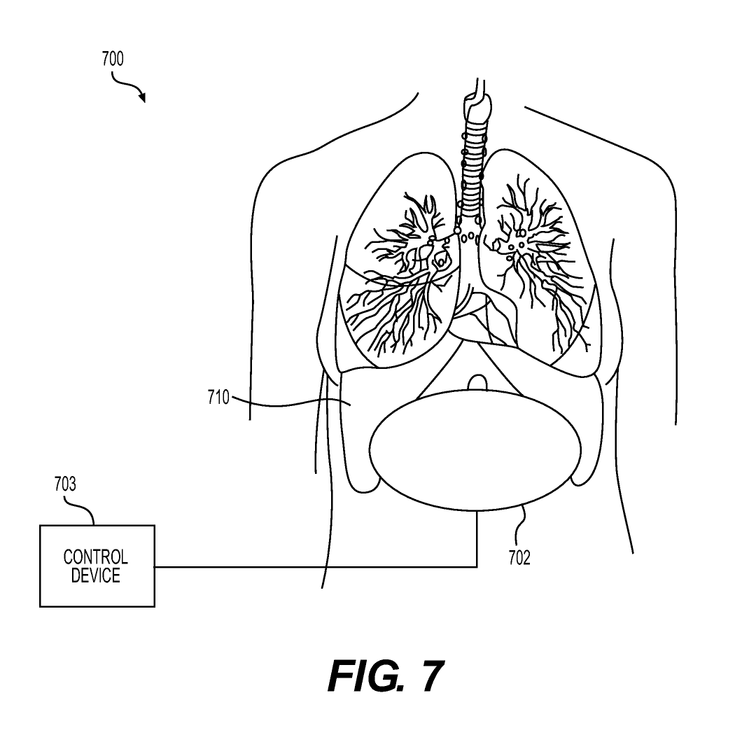

FIG. 7 illustrates another alternative embodiment in which an exemplary expandable system 702 is deployed to boost or otherwise assist a diaphragm's 710 ability to expand according to various embodiments of the present disclosure. The expandable system 702 may expand during exhalation and may apply outward (relative to expandable system 702) radial force to boost the diaphragm's 710 ability to expand. During inhalation by a healthy patient, the diaphragm 710 contracts thus enlarging the volume of the thoracic cavity. Further, the enlargement of the thoracic cavity creates suction that draws air into the lungs. When the diaphragm 710 relaxes, the air is exhaled by elastic recoil of the lungs and the tissue lining the thoracic cavity.

In at least one embodiment, the expandable system 702 may be a boost device. In a patient suffering from COPD, the expandable system 702 (e.g., the boost device) may be used to assist the diaphragm 710 during respiratory functions. For example, during inhalation, the expandable system 702, e.g. the boost device, may collapse so as to permit contraction of the diaphragm 710. The boost device may assist the exhalation function and overcome the less flexible alveoli to exhale trapped air.

The expandable system 702 may detect the breathing function and synchronize with the diaphragm 710 to deliver the boost function during exhalation. In some embodiments, the expandable system 702 may include built in logic features that may detect and record, or otherwise learn, a patient's breathing patterns. For example, the expandable system 702 may include one or more electrical and/or pressure sensors configured to measure respiration of a patient. In at least one embodiment, and in order to remain in sync with the breathing cycle of the diaphragm 710, the expandable system 702 may include a material sensitive to changes in pressure that may constrict or collapse in upon itself during inhalation. During exhalation, the change in pressure may cause the expandable system 702 to expand. Accordingly, the expandable system 702 may be configured to automatically remain in sync with the diaphragm's 710 contraction and expansion cycles.

Further, the expandable system 702 may deliver sufficient radial force to the inner diaphragm 710 walls to overcome the opposing mass of the damaged tissue of the lung system 700 during exhalation. While depicted in FIG. 7 as positioned immediately beneath diaphragm 710, expandable system, 702 may be positioned anywhere within the patient's body, e.g., in the pleural cavity adjacent a side of a lung. The expandable system 702 may have any suitable form including, but not limited to, a self-expanding stent, compression spring, or balloon-like pouch.

In some embodiments, the expandable system 702 may remain connected to an external device, such as, a control device 703 (or controller). The control device 703 may control contraction or expansion of the expandable system 702 (e.g., removal and/or delivery of fluid within expandable system 702) by sensing of various signals such as, for example, voltage, pressure, and/or impedance. The controller 703 may be configured to sense a biological event and control an amount of outwardly directed force applied by the expandable system 702 to the lung in response to a biological event. Examples of biological events may include, but are not limited to, dyspnea or an irregular heartbeat, temperature, etc. The controller 703 also may be configured to decrease the amount of outwardly directed force applied by the expandable system 702 to the lung after the cessation of the biological event. In some embodiments, the biological event is one of an inhalation and exhalation rhythm of the lung. The controller 703 further can be configured to synchronize the outwardly directed force applied by the expandable system 702 to the lung by one of the inhalation and exhalation rhythm(s). The controller 703 can be configured to increase the outwardly directed force applied by the expandable system 702 to the lung during exhalation and/or to decrease the outwardly directed force applied by the expandable system 702 to the lung during inhalation. The expandable system 702 may be active or passive in that it may or may not utilize an external controller 703 or be self-regulating in order for its activation to be in sync with the movement of the diaphragm 710.

In some embodiments, the expandable system 702 may be manufactured from an electro-active polymer. When electrical voltage is applied, the expandable system 702 may expand to assist with exhalation. When voltage is removed, the expandable system 702 may relax and be caused to contract.

In an alternate embodiment, the expandable system 702 can be formed as a spring. The spring may convert the force of the lung tissue mass pressing down on the diaphragm 710 during inhalation from potential energy into kinetic energy to boost the diaphragm 710 during exhalation. For example, the mass of the lung tissue during inhalation may compress the spring. Then, the spring may apply this force to the inner walls of the diaphragm 710 during exhalation. The spring may be configured to apply sufficient outward force to overcome the opposing mass of the damaged lung tissue during exhalation, but not enough to impede on the diaphragm's 710 ability to constrict during inhalation. The expandable system 702 may be anchored or attached to the inner walls of the diaphragm 710 through the use of adhesives or embedment into the walls of diaphragm 710. This expandable system 702 may be active or passive with the use of electrical or heat reactive polymers or metals (such as, Nitinol).

In yet another embodiment, the expandable system 702 can include a pouch or balloon device that may expand during exhalation, applying outward, radial force to boost the diaphragm's 710 ability to expand. During inhalation, the balloon/pouch may collapse to permit the contraction of the diaphragm 710. In order for the balloon/pouch to remain in sync with the breathing cycle of the diaphragm 710, the balloon/pouch may feature a material sensitive to changes in pressure that may constrict or collapse in upon itself during inhalation (e.g., piezo-metallic and/or pressure sensitive adhesive materials). During exhalation, the change in pressure may cause the balloon/pouch to inflate in order to remain in sync with the diaphragm's 710 contraction and expansion cycles. The balloon/boost device 702 may be contoured to mimic the shape of the diaphragm 710 or may have a simple, rounded shape during expansion as shown in FIG. 7.

Another embodiment of the present disclosure may include the use of a self-expanding stent to boost the diaphragm 710 during exhalation. The stent may be formed using suitable polymers or advanced metals (e.g., Nitinol). Further, the stent may be self-regulating and may not require any external control (for example, controller 703) to sync to the diaphragm 710. Further, the stent may be configured to collapse during inhalation so as to allow normal functioning of the diaphragm 710. Further, natural expansion and contraction of the stent may be synchronized with that of the diaphragm 702. In addition, the radial outward force of the stent may push against in the inner walls of the diaphragm 702 to support exhalation.

The embodiments of the expandable system 702 as described above may require constant contact with the inner walls of the diaphragm 710. Accordingly, the expandable system 702 also may include anti-migration features that may remain in constant contact with the inner walls of the diaphragm 710 to ensure the device remains in sync with the diaphragm 710 during inhalation and exhalation. These anti-migration features may also ensure that the expandable system 702 may not become dislodged or move relative to the diaphragm 710. The anti-migration features may include textures having tacky surfaces to ensure that the expandable system 702 (having a boost device) remains attached to the inner diaphragm's 710 walls. Other anti-migration alternatives such as adhesives or small barbs that can fix to the inner walls of the diaphragm 710 also may be used. It is understood that any of the disclosed anti-migration features may be used with any of the disclosed embodiments of the expandable system (e.g., 102, 302, 502, 602, and 702) discussed herein.

Embodiments of the present disclosure also provide a method for expelling air from the lung or evacuating a cavity by applying a force to the lung during exhalation via an expandable element placed in the lung cavity to expel air from the lungs. The method may include inserting an expandable system (such as expandable systems 102, 302, 502, 602, and 702). Further, the expandable system may be a balloon filled with suitable fluid (e.g., dilatant fluid), a pouch, a sponge, a boost device, a stent, a spring system, a polymer, and so forth. Then, the expandable system may be sized appropriately based on the size/shape/nature of the damaged tissue (e.g. tissue 106, 306, etc.). In some embodiments, the expandable system may be sized in accordance with breathing cycles of the patient or by injecting more material into the internal volume of the expandable system by an operator. The operator may achieve this via a suitable access port, which may be disposed under the skin at a location remote from the expandable system (such as expandable systems 102, 302, 502, 602, and 702) but operably coupled to the interior of the expandable system (such as expandable systems 102, 302, 502, 602, and 702).

Once implanted, the expandable system may contract during inhalation and expand during exhalation. The expandable system may apply a force to the lung during exhalation to expel air from the lung. In some embodiments, the expansion or contraction of the expandable system may be controlled using a controller (e.g., controller 703) as mentioned above. Further, an electrical charge may be applied by the controller to cause the expandable system to transit from a first configuration to a second configuration. The electrical charge may be applied, for example, during exhalation of the lung. The electrical charge may be applied via an operably coupled implanted pulse generator. In some embodiments, the expandable system can be retracted (e.g., collapsed) in the first configuration and expanded in the second configuration. In some embodiments, the expandable system may include an electro-active material that is configured to collapse based on the variation in electric charge by the controller.

In embodiments without a controller, the expandable system such as the balloon, sponge etc. may regain its original shape and size post exhalation as a result of native resilient properties. In such embodiments, the expandable system may include a pressure sensitive material that is configured to collapse during inhalation of the lung.

Further exemplary embodiments of the present disclosure include embodiments directed to a filler implant or expandable system which may be placed adjacent to a target area, e.g., an area in the lung system where air bullae (e.g., blisters or elevated lesions) may have appeared due to loss of elasticity and lung inability to passively recoil during expiration as the diaphragm and intercostal muscles relax. Due to this loss of elasticity, the air is not adequately pushed out of the lung system. The target area may be treated through lung volume reduction using a filler implant. The filler implant can be configured to apply compression on the lung(s).

In at least one embodiment, the filler implant can be a passive implant that may resemble the implants used for breast augmentation/reconstruction. In some embodiments, the passive implant may include an outer shell made of pebax or silicone which may be inserted empty, between the lung and the rib cage. The filler implant then may be filled with sterile salt water or any suitable materials, which can compress the lung and prevent the lung from overfilling. The filler implant and the sterile salt water (dilatant fluid or other suitable fluid) may exert a force on the damaged tissue during exhalation of the lung. In another embodiment, the passive implant includes silicone gel that can be implanted between the lungs and the rib cage.

In another embodiment, the filler implant can be a semi-smart implant that resembles those used for breast augmentation or reconstruction. The semi-smart implant may include an outer shell filled with non-Newtonian fluid that when under shear stress changes its viscosity for a while (thinning or thickening, depending on its thixotropic or dilatant nature). The semi-smart implant can be responsive to physiotherapy.

In yet another embodiment, the filler implant can be a smart implant or including an electronic mechanism like a pacemaker, implanted between the pleurae (lung) and the rib cage. Under certain circumstances, the mechanism may initiate a cycle during which the implant may deploy itself by taking up more volume and compressing against the lung to push trapped air out. Examples of the circumstances in which the mechanism may initiate include, but are not limited to, a signal from a physiotherapist emptying the lung under a controlled environment, shortness of breath (i.e. dyspnea), irregular heartbeat, or initial stages or exacerbation. Further, the mechanism may be initiated by the patient at regular intervals.

In further embodiments, the filler implant may include an outer silicone shell or pebax balloon which may be inserted empty between lung and rib cage, with a chest tube cannula and a port-valve attached. The shell/balloon can remain implanted. Further, the shell/balloon may be filled or emptied periodically using an external pump that the patient can wear in a belt or in a holster, etc.

Although the exemplary embodiments described above have been disclosed in connection with medical devices for insertion into a lung cavity for effective expelling of the air from the lung, those skilled in the art will understand that the principles set out above can be applied to any bronchial device and can be implemented in different ways without departing from the scope of the disclosure as defined by the claims. In particular, constructional details, including manufacturing techniques and materials, are well within the understanding of those of skill in the art and have not been set out in any detail here. These and other modifications and variations are well within the scope of the present disclosure and can be envisioned and implemented by those of skill in the art.

Other exemplary embodiments of the present disclosure will be apparent to those skilled in the art from consideration of the specification and practice of the exemplary embodiments disclosed herein. It is intended that the specification and examples be considered as exemplary only, and departures in form and detail may be made without departing from the scope and spirit of the present disclosure as defined by the following claims.

What is claimed is:

1. A method for assisting breathing in a lung having damaged tissue, the method comprising:
inserting an implant containing a dilatant fluid into a pleural cavity of a patient to reduce an inhalation volume of the lung.

2. The method of claim 1, wherein the damaged tissue is an emphysematous portion of the lung.

3. The method of claim 1, further including inserting the implant adjacent to the damaged tissue to prevent the damaged tissue from expanding during inhalation.

4. The method of claim 1, wherein the implant and dilatant fluid exert a force on the damaged tissue during exhalation of the lung.

5. The method of claim 1, wherein the dilatant fluid includes one or more of hyaluronic acid, lubricin, cornstarch, water, Dilatal, acrylic acid, ester-styrene, silicone oil, boric acid, and ethylene glycol.

6. A system, comprising:
an implant for insertion into a lung cavity, the implant being configured to apply an outwardly directed force toward a portion of a lung disposed in the lung cavity; and
a controller coupled to the implant, the controller being configured to sense a biological event and control an amount of outwardly directed force applied by the implant to the lung in response to the biological event.

7. The medical device of claim 6, wherein the biological event is dyspnea or an irregular heartbeat.

8. The medical device of claim 6, wherein the controller is further configured to increase the amount of outwardly directed force applied by the implant to the lung in response to the biological event.

9. The medical device of claim 8, wherein the controller is further configured to decrease the amount of outwardly directed force applied by the implant to the lung after the cessation of the biological event.

10. The medical device of claim 6, wherein the biological event is one of an inhalation and exhalation rhythm of the lung.

11. The medical device of claim 10, wherein the controller is further configured to synchronize the outwardly directed force applied by the implant to the lung by the one of the inhalation and exhalation rhythm.

12. The medical device of claim 10, wherein the controller is configured to increase the outwardly directed force applied by the implant to the lung during exhalation of the lung.

13. The medical device of claim 10, wherein the controller is configured to decrease the outwardly directed force applied by the implant to the lung during inhalation of the lung.

14. The medical device of claim 6, wherein the implant includes an anti-migration element.

15. A method of assisting in evacuating an interior volume of an organ, the method comprising:
applying a force to the organ via an adjustable implant placed adjacent the organ to assist in avacuating material from the interior volume of the organ; and
adjusting the implant so as to transition between a first configuration and a second configuration;
wherein the implant includes at least one of an electro-active material and a pressure sensitive material.

16. The method of claim 15, wherein if the implant includes an electro-active material, further including:
applying an electrical charge to cause the implant to transition between the first and second configurations.

17. The method of claim 15, wherein the implant is collapsed in the first configuration and is expanded in the second configuration.

18. The method of claim 16, wherein the organ is a lung and the material is a fluid, and wherein the electrical charge is applied during exhalation of the lung.

19. The method of claim 15, wherein if the implant includes a pressure sensitive material, wherein the organ is a lung and the material is air, and wherein the pressure sensitive material is configured to collapse during inhalation of the lung and expand during exhalation of the lung.

20. The method of claim 15, wherein the implant further includes a spring, a stent, or a polymer.
